(19) United States
(12) Patent Application Publication
(10) Pub. No.: US 2007/0101447 A1
(43) Pub. Date: May 3, 2007

(54) Diagnostic tests for the detection of motor neuropathy

(75) Inventors: Peter De Jonghe, Mechelen (BE); Vincent Timmerman, Broechem (BE); Joy Irobi, Edegem (BE); Christine Van Broeckhoven, Edegem (BE)

Correspondence Address: TRASK BRITT, P.O. BOX 2550, SALT LAKE CITY, UT, 84110, US

(73) Assignee: Vlaams Interuniversitair Instituut Voor Biotechnologie VZW, Zwijnaarde (BE)

(21) Appl. No.: 10/579,456

(22) PCT Filed: Nov. 15, 2004
(86) PCT No.: PCT/EP04/52962

§ 371 Date: Dec. 8, 2006

(30) Foreign Application Priority Data
Nov. 13, 2003 (EP) 03104181.7

(51) Int. Cl. A01K 67/00; C12Q 1/68; C07H 21/04; A61K 38/17

(52) U.S. Cl. 800/8; 435/6; 435/320.1; 435/325; 536/23.5; 514/12

(57) ABSTRACT

The present invention relates generally to the field of human genetics. Specifically, the present invention relates to methods and materials used to diagnose distal hereditary motor neuropathy type II by detecting polymorphisms in HSP22 gene.

### FIELD OF THE INVENTION

The present invention relates generally to the field of human genetics. Specifically, the present invention relates to methods and materials used to isolate and detect human motor neuron diseases causing genes, some alleles of which cause motor neuron neuropathy.

### BACKGROUND OF THE INVENTION

Hereditary peripheral neuropathies, among the most common genetic disorders in humans, are a complex, clinically and genetically heterogeneous group of disorders and they produce progressive deterioration of the peripheral nerves. This group of disorders includes hereditary motor and sensory neuropathies (HMSN), hereditary motor neuropathies (HMN) and hereditary sensory neuropathies (HSN). Our understanding of these disorders has progressed from the description of the clinical phenotypes and delineation of the electrophysiologic and pathologic features to the identification of disease genes and elucidation of the underlying molecular mechanisms. Recent molecular analysis of the inherited peripheral neuropathies (IPN) has led to important insights into the process of myelination and the function of some of the genes involved. An important problem for the physician is that the IPN show considerable clinical and genetical heterogeneity. The discovery that mutations in multiple genes result in similar phenotypes argues for complex protein interactions and complementing functions for each protein product within the myelin sheath. Knowledge of the structure and function of the causal genes is currently being actively pursued to better classify peripheral neuropathies and to elucidate the underlying molecular mechanisms of these diseases. Thus, the knowledge of the exact genetic aberration in the patients has important ramifications for diagnosis, prognosis, genetic counseling, and approaches for therapy. In the present invention we have identified missense mutations in the heat shock protein HSP22 associated with a motor neuron disease, more particularly distal hereditary motor neuron disease type II. Distal HMN is genetically heterogeneous and so far, 12 loci and 7 genes have been reported for distal HMN²⁻⁴ and Irobi J. et al (2004) Human Mol. Gen. 13, 195). The present invention can be used for the manufacture of a diagnostic assay for a more correct diagnosis of motor neuron diseases.

### FIGURE LEGENDS

FIG. 1: Segregation of K141N, K141E and R78M mutations in HSP22 in five distal HMN II pedigrees.

(a) Belgian CMT-M family, (b) Bulgarian AJ-12 family, (c) Czech CMT-196 family and (d) English CMT-355 family, e) Bulgarian AJ-135.

Symbols: squares=male, circles=female, filled symbol=affected, empty symbol=unaffected, slashed symbol=deceased, and half symbol=unknown clinical status.

FIG. 2: Refinement and segregation of 12q24.3 STR markers in the Czech and Belgian families (CMT-M and CMT-196).

Symbols: squares=male, circles=female, filled symbol=affected, empty symbol=unaffected, the alleles represent the allele size in bp obtained with the STR marker, - - =failed genotypes. The distal HMN II locus was refinement from 5 Mb⁷ to 1.7 Mb. Comparison of haplotypes in the Czech, Belgian, and the Bulgarian, English families. The disease haplotype is boxed in blue (Belgian and Czech), red (Bulgarian, English) and the shared alleles are shown in bold.

FIG. 3: DNA and protein sequence analysis of HSP22.

(a) Electropherogram showing the c.421A>G and c.423G>C sequence variations in HSP22 exon 2 resulting in the K141E and K141N missense mutations in families AJ-12, CMT-355, CMT-M and CMT-196. The corresponding genomic sequence of a control person is shown in the middle. (b) The HSP22 gene spans˜15 kb and contains 3 exons, the α-crystallin domain is indicated in red. (c) ClustalW multiple protein alignment (http://npsa-pbil.ibcp.fr/cgi-bin/npsa_automat.pl?page=npsa_clustalw.html) of Hsp22 orthologues. The protein sequences show the α-crystallin domain (fragment 120-145) surrounding the K141E and K141N mutations. Hsp22 orthologues: Human (Homo. sapiens), mouse (Mus musculus), rat (Rattus norvegicus), fly (Drosophila melanogaster), bacteria (Mycobacterium leprae), nematode (Caenorhabditis elegans), and bread wheat, (Triticum aestivum). The N-myristoylation site is boxed in green and the PKC phosphorylation site; N-glycosylation motif is boxed. Both amino acid mutations are indicated by an arrow.

FIG. 4: Sequence of HSP22.

(a) SEQ ID NO. 1: Nucleotide sequence of HSP22, Homo sapiens

(b) SEQ ID NO. 2: Amino acid sequence of HSP22, Homo sapiens

FIG. 5: Detected SNPs are indicated with arrows. Coding regions are boxed in dark; TATA boxes are boxed in white; HSE=heat shock elements.

### AIMS AND DETAILED DESCRIPTION OF THE INVENTION

The present invention relates generally to the field of human genetics. Specifically, the present invention relates to methods and materials used to isolate and detect a human motor neuron disease causing gene (HSP22), some alleles of which cause motor neuron disease. In particular alleles of HSP22 cause distal hereditary motor neuron disease type II. HSP22 belongs to the superfamily of mammalian small heat shock proteins (sHSPs) or stress proteins, which in humans consists of the HSP27, alpha A and B-crystallin, HSP20, HSPB2, HSPB3 and cvHSP¹¹⁻¹². Several cellular functions have been attributed to sHSPs. They are able to protect cells from stress conditions by preventing both apoptosis and necrosis^{13,14}. Throughout the animal, plant, and microbiotic kingdoms, members of this protein super-family share a conserved α-crystallin domain in their C-terminal part, and the WDPF motif in their N-terminal part whereas other parts of the sequence (N-terminal halves and extreme C-terminal tails) are more variable^{15,16}.

“Motor neuron disease” can be defined as a group of diseases involving the degeneration of the anterior horn cells, nerves in the central nervous system that control muscle activity. This leads to gradual weakening and eventually wasting of the musculature (atrophy). Diseases of the motor neuron are classified according to upper motor neuron (UMN) and/or lower motor neuron (LMN) involvement. Upper motor neurons originate in the brain, in particular, the motor cortex, and they synapse either directly or indirectly onto lower motor neurons. Upper motor neurons are more accurately referred to as pre-motor neurons, and they are responsible for conveying descending commands for movement. Lower motor neurons are dividable into two categories: visceral and somatic motor neurons. Visceral motor neurons are autonomic pre-ganglionic neurons that regulate the activity of ganglionic neurons, which innervate glands, blood vessels, and smooth muscle. Somatic motor neurons innervate skeletal muscle and include first, anterior horn cells, which as the name implies, are located in the anterior horn of the spinal cord, and second, lower motor neurons located in the cranial nerve nuclei. Amyotrophic lateral sclerosis or ALS is the most frequent form (accounting for around 80% of all cases) of motor neuron disorders. ALS is known as Lou Gehrig's disease, named after the famous Yankee baseball player. The initial symptoms of ALS are weakness in the hands and legs and often fasciculation of the affected muscles. Whichever limbs are affected first, all four limbs are affected eventually. Damage to the upper motor neurons produces muscle weakness, spasticity and hyperactive deep tendon reflexes. Lower motor neuron damage produces muscle weakness with atrophy, fasciculations, flaccidity and decreased deep tendon reflexes. ALS has features of both upper and lower motor neurons of the cranial nerves, therefore symptoms are isolated to the head and neck. Some patients will also display UMN involvement of the cranial nerves and if this is the sole manifestation it is referred to as Pseudobulbar pulsy. Spinal muscular atrophy or progressive muscular atrophy is a MND that does not involve the cranial nerves and is due to lower motor neuron degeneration. Shy-Drager syndrome is characterized by postural hypotension, incontinence, sweating, muscle rigidity and tremor, and by the loss of neurones from the thoracic nuclei in the spinal cord from which sympathetic fibres originate. Destructive lesions of the spinal cord result in the loss of anterior horn cells. This is seen in myelomeningocele and in syringomyelia, in which a large fluid-filled cyst forms in the centre of the cervical spinal cord. Poliomyelitis virus infection also destroys anterior horn cells. Spinal cord tumours may locally damage anterior horn cells either by growth within the cord (gliomas) or by compression of the spinal cord from the outside (meningiomas, schwannomas, metastatic carcinoma, lymphomas). Dorsal root ganglion cells may be damaged by herpex simplex and varicella-zoster viruses. Such infections are associated with a vesicular rash in the skin regions supplied by those neurones. A similar loss of sensory neurones is observed in ataxia telangiectasia, a disorder associated with progressive cerebellar ataxia and symmetrical telangiectases of the skin and conjunctiva. Neuronal loss from autonomic ganglia is observed in amyloid neuropathies and in diabetes. “Hereditary motor neuron disease type II” can be defined as an exclusive lower motor neuron disease without sensory loss and is also designated distal HMNII. The presenting symptoms are paresis of the extensor muscles of the big toe and later of the extensor muscles of the feet. The disease progresses rapidly and within 5 years a complete paralysis of all distal muscles of the lower extremities occurs.

Thus the invention discloses methods for determining the presence or absence of HSP22 mutations, which are useful in the diagnosis of HMNII. Mutations of HSP22 (SEQ ID NO: 1) causing motor neuron disease and more particularly HMNII are included in Table 1. The amino acid sequence of HSP22 is depicted in SEQ ID NO: 2. These nucleic acids or fragments capable of specifically hybridizing with the corresponding allele in the presence of other HSP22 alleles under stringent conditions find broad diagnostic application. Gene products of the disclosed mutant HSP22 alleles also find a broad range of diagnostic applications. For example, mutant allelic HSP22 peptides can be used to generate specific binding compounds. Binding reagents can be used diagnostically to distinguish wild-type and HMNII causing HSP22 translation products. The subject nucleic acids (including fragments thereof) may be single or double stranded and are isolated, partially purified, and/or recombinant. An “isolated” nucleic acid is present as other than a naturally occurring chromosome or transcript in its natural state and isolated from (not joined in sequence to) at least one nucleotide with which it is normally associated on a natural chromosome; a partially pure nucleic acid constitutes at least about 10%, preferably at least about 30%, and more preferably at least about 90% by weight of total nucleic acid present in a given fraction; and a recombinant nucleic acid is joined in sequence to at least one nucleotide with which it is not normally associated on a natural chromosome.

In a first embodiment the invention provides an isolated nucleic acid coding for a dominant negative, mutant HSP22 polypeptide, said nucleic acid containing in comparison to the wild type HSP22 encoding sequence set forth in SEQ ID NO: 1 one or more mutations wherein the presence of said nucleic acids is indicative for the presence of a motor neuron disease and more particularly for the presence of HMNII.

In yet another embodiment the invention provides an isolated nucleic acid coding for a dominant negative, mutant HSP22 polypeptide, said nucleic acid containing in comparison to the wild type HSP22 encoding sequence set forth in SEQ ID NO: 1 one or more mutations selected from the mutations set forth in Table 1 wherein the presence of said nucleic acids is indicative for the presence of a motor neuron disease and more particularly for the presence of HMNII.

‘Mutant’ as used herein refers to a gene that encodes a mutant protein. With respect to proteins, the term “mutant” means a protein which does not perform its usual or normal physiological role and which is associated with, or causative of, a pathogenic condition or state. Therefore, as used herein, the term “mutant” is essentially synonymous with the terms “dysfunctional,” “pathogenic,” “disease-causing,” and “deleterious.” With respect to the gene encoding HSP22 protein of the present invention, the term “mutant” refers to a gene encoding HSP22, bearing one or more nucleotide/amino acid substitutions, insertions and/or deletions which for example can lead to the development of the symptoms of a motor neuron disorder when expressed in humans. This definition is understood to include the various mutations that naturally exist, including but not limited to those disclosed herein, as well as synthetic or recombinant mutations produced by human intervention. The term “mutant,” as applied to the gene encoding HSP22, is not intended to embrace sequence variants which, due to the degeneracy of the genetic code, encode proteins identical to the normal sequences disclosed or otherwise presented herein; nor is it intended to embrace sequence variants which, although they encode different proteins, encode proteins which are functionally equivalent to normal HSP22. Assays to measure the activity of (mutant) HSP22-proteins are disclosed in the art (e.g. MTS assay: (http://iprotocol.mit.edu/protocol/281.htm)). Said assays can for example be used to measure a possible dominant effect of the identified HSP22-mutations in patients suffering from a motor neuron disorder. A ‘dominant negative’ allele or a ‘dominant negative’ gene is a mutant allele or mutant gene that when inherited it manifests the phenotype of the mutation even in the presence of a wild type allele or gene.

In another embodiment the invention provides a nucleic acid probe wherein the nucleotide sequence is a fragment of a nucleic acid sequence derived from a dominant negative, mutant HSP22 gene.

As used herein, “fragment” refers to a nucleotide sequence of at least about 9 nucleotides, typically 15 to 75, or more, wherein said nucleotide sequence comprises at least one mutation for HSP22.

In another embodiment the isolated nucleic acids of the present invention include any of the above described sequences or fragments thereof of HSP22 when included in vectors. Appropriate vectors include cloning vectors and expression vectors of all types, including plasmids, phagemids, cosmids, episomes, and the like, as well as integration vectors. The vectors may also include various marker genes (e.g., antibiotic resistance or susceptibility genes) that are useful in identifying cells successfully transformed therewith. In addition, the vectors may include regulatory sequences to which the nucleic acids of the invention are operably joined, and/or may also include coding regions such that the nucleic acids of the invention, when appropriately ligated into the vector, are expressed as fusion proteins. Such vectors may also include vectors for use in yeast “two hybrid,” baculovirus, and phage-display systems. The vectors may be chosen to be useful for prokaryotic, eukaryotic or viral expression, as needed or desired for the particular application. For example, vaccinia virus vectors or simian virus vectors with the SV40 promoter (e.g., pSV2), or Herpes simplex virus or adeno-associated virus may be useful for transfection of mammalian cells including dorsal root ganglia or neurons in culture or in vivo, and the baculovirus vectors may be used in transfecting insect cells. A great variety of different vectors are now commercially available and otherwise known in the art, and the choice of an appropriate vector is within the ability and discretion of one of ordinary skill in the art.

In yet another embodiment the invention provides a host cell comprising a recombinant vector according to the invention.

In yet another embodiment the invention provides a method for the preparation of a diagnostic assay to detect the presence of a motor neuron disorder in a human comprising detecting at least one mutation in the nucleotide position of SEQ ID NO: 1 in a tissue sample of said human, wherein said mutation respectively results in a dominant mutation of HSP22 and wherein the presence of said mutation is indicative of the presence of a motor neuron disorder and more particularly for the presence of HMNII.

In yet another embodiment the invention provides a diagnostic method for determining if a subject bears a mutant gene encoding HSP22 comprising the steps of (1) providing a biological sample of said subject, and (2) detecting in said sample a mutant nucleic acid encoding a HSP22 protein or a mutant HSP22 protein activity.

The HSP22 gene and gene product, as well as other products derived thereof (e.g., probes, antibodies), can be useful in the diagnosis of motor neuron disorders, and probably also in acquired forms of motor neuron disorders (in other words to detect if a human has a predisposition to acquire a motor neuron disorder or more particularly HMNII). Diagnosis of for example inherited cases of these diseases can be accomplished by methods based upon the nucleic acids (including genomic and mRNA/cDNA sequences), proteins, and/or antibodies. Preferably, the methods and products are based upon the human HSP22 gene, protein or antibodies against the HSP22 protein. As will be obvious to one of ordinary skill in the art, however, the significant evolutionary conservation of large portions of HSP22 nucleotide and amino acid sequences, even in species as diverse as humans and C. elegans and Drosophila, allow the skilled artisan to make use of such non-human HSP22-homologue nucleic adds, proteins and antibodies even for applications directed toward human or other mammalian subjects. Thus, for brevity of exposition, but without limiting the scope of the invention, the following description will focus upon uses of the human homologues of HSP22 genes and proteins. It will be understood, however, that homologous sequences from other species will be equivalent for many purposes. As will be appreciated by one of ordinary skill in the art, the choice of diagnostic methods of the present invention will be influenced by the nature of the available biological samples to be tested and the nature of the information required. The HSP22 gene is highly expressed in different parts of the brain, the spinal cord, dorsal root ganglia (sensory neurons) and the ventral horn (motor neurons). Brain or spinal cord or motor neuron biopsies are invasive, dangerous, difficult and expensive procedures, particularly for routine screening. Other tissues, which express the HSP22 gene at significant levels, however, may demonstrate alternative splicing (e.g., white blood cells) and, therefore mRNA derived from HSP22 gene or proteins from such cells may be less informative. Thus, assays based upon a subject's genomic DNA may be the preferred methods for diagnostics of HSP22 gene as no information will be lost due to alternative splicing and because essentially any nucleate cells may provide a usable sample. When the diagnostic assay is to be based upon nucleic acids from a sample, either mRNA or genomic DNA may be used. When mRNA is used from a sample, many of the same considerations apply with respect to source tissues and the possibility of alternative splicing. That is there may be little or no expression of transcripts unless appropriate tissue sources are chosen or available, and alternative splicing may result in the loss of some information. With either mRNA or DNA, standard methods well known in the art may be used to detect the presence of a particular sequence either in situ or in vitro (see, e.g. Genome Analysis, A laboratory Manual, eds E. D. Green, B. Birren, S. Klapholz, R. M. Myers, P. Hieter, Cold Spring Harbor Laboratory Press, 1997). In a preferred embodiment of the invention, the starting nucleic acid represents a sample of DNA isolated from an animal or human patient. This DNA may be obtained from any cell source or body fluid. Non-limiting examples of cell sources available in clinical practice include blood cells, buccal cells, cervico-vaginal cells, epithelial cells from urine, or any cells present in tissue obtained by biopsy. Body fluids include blood, urine, and cerebrospinal fluid. DNA is extracted from the cell source or body fluid using any of the numerous methods that are standard in the art. It will be understood that the particular method used to extract DNA will be chosen as being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction and/or phenol extractions can be used to obtain nucleic acid from cells or tissues, e.g., blood. In a specific embodiment, the cells may be directly used without purification of the target nucleic acid. For example, the cells can be suspended in hypotonic buffer and heated to about 90-100° C., until cell lysis and dispersion of intracellular components occur, generally about 1 to 15 minutes. After the heating step the amplification reagents may be added directly to the lysed cells. This direct amplification method may for example be used on peripheral blood lymphocytes. The preferred amount of DNA to be extracted for analysis of human genomic DNA is at least 5 pg (corresponding to about 1 cell equivalent of a genome size of 4.10⁹ base pairs). In some applications, such as, for example, detection of sequence alterations in the genome of a microorganism, variable amounts of DNA may be extracted. In a particular embodiment, the starting nucleic acid is RNA obtained, e.g., from a cell or tissue. RNA can be obtained from a cell or tissue according to various methods known in the art and described, e.g. Genome Analysis, A laboratory Manual, eds E. D. Green, B. Birren, S. Klapholz, R. M. Myers, P. Hieter, Cold Spring Harbor Laboratory Press, 1997. For in situ detection of a mutant nucleic acid sequence of HSP22, a sample of tissue may be prepared by standard techniques and then contacted with a probe, preferably one which is labeled to facilitate detection, and an assay for nucleic acid hybridization is conducted under stringent conditions which permit hybridization only between the probe and highly or perfectly complementary sequences. In many applications, the nucleic acids are labeled with directly or indirectly detectable signals or means for amplifying a detectable signal. Examples include radiolabels, luminescent (e.g. fluorescent) tags, components of amplified tags such antigen-labeled antibody, biotin-avidin combinations etc. The nucleic acids can be subject to purification, synthesis, modification, sequencing, recombination, incorporation into a variety of vectors, expression, transfection, administration or methods of use disclosed in standard manuals such as Genome Analysis, A laboratory Manual, eds E. D. Green, B. Birren, S. Klapholz, R. M. Myers, P. Hieter, Cold Spring Harbor Laboratory Press, 1997 or that are otherwise known in the art. Because many mutations in genes that cause diseases detected to date consist of a single nucleotide substitution, high stringency hybridization conditions will be required to distinguish normal sequences from most mutant sequences. A significant advantage of the use of either DNA or mRNA is the ability to amplify the amount of genetic material using the polymerase chain reaction (PCR), either alone (with genomic DNA) or in combination with reverse transcription (with mRNA to produce cDNA). Other nucleotide sequence amplification techniques may be used, such as ligation-mediated PCR, anchored PCR and enzymatic amplification as will be understood by those skilled in the art. Sequence alterations may also generate fortuitous restriction enzyme recognition sites, which are revealed by the use of appropriate enzyme digestion, followed by gel-blot hybridization. DNA fragments carrying the site (normal or mutant) are detected by their increase or reduction in size, or by the increase or decrease of corresponding restriction fragment numbers. Genomic DNA samples may also be amplified by PCR prior to treatment with the appropriate restriction enzyme and the fragments of different sizes are visualized, for example under UV light in the presence of ethidium bromide, after gel electrophoresis. Genetic testing based on DNA sequence differences may be achieved by detection of alteration in electrophoretic mobility of DNA fragments in gels. Small sequence deletions and insertions can be visualized by high-resolution gel electrophoresis of single stranded DNA, or as changes in the migration pattern of DNA heteroduplexes in non-denaturing gel electrophoresis. Alternatively, a single base substitution mutation may be detected based on differential PCR product length in PCR. The PCR products of the normal and mutant gene may be differentially detected in acrylamide gels. Nuclease protection assays (S1 or ligase-mediated) also reveal sequence changes at specific locations. Alternatively, to confirm or detect a polymorphism resulting in restriction mapping changes. Ligated PCR, allele specific oligonucleotide probes (ASOs), REF-SSCP chemical cleavage, endonuclease cleavage at mismatch sites or SSCP may be used. Both REF-SSCP and SSCP are mobility shift assays, which are based upon the change in conformation due to mutations. DNA fragments may also be visualized by methods in which the individual DNA samples are not immobilized on membranes. The probe and target sequences may be in solution or the probe sequence may be immobilized. Autoradiography, radioactive decay, spectrophotometry and fluorometry may also be used to identify specific individual genotypes. Mutations in HSP22 can also be detected by direct nucleotide sequencing. Methods for nucleotide sequencing are well known in the art. Fragments of the disclosed alleles of HSP22 are sufficiently long for use as specific hybridization probes for detecting endogenous alleles, and particularly to distinguish the disclosed mutant alleles, which correlate with motor neuron diseases, more particularly HMNII. Preferred fragments are capable of hybridizing to the corresponding mutant allele under stringency conditions characterized by a specific hybridization buffer. In any event, the fragments are necessarily of length sufficient to be unique to the corresponding allele; i.e. has a nucleotide sequence at least long enough to define a novel oligonucleotide, usually at least about 14, 16, 18, 20, 22, or 24 bp in length, though such fragment may be joined in sequence to other nucleotides which may be nucleotides which naturally flank the fragment. For example, where the subject nucleic acids are used as PCR primers or hybridization probes the subject primer or probe comprises an oligonucleotide complementary to a strand of the mutant or rare allele of length sufficient to selectively hybridize with the mutant or rare allele. Generally, these primers and probes comprise at least 16 bp to 24 bp complementary to the mutant or rare allele and may be as large as is convenient for the hybridizations conditions. In some cases where the critical mutation in HSP22 is a deletion of wild-type sequence, useful primers/probes require wild-type sequences flanking (both sides) the deletion with at least 2, usually at least 3, more usually at least 4, most usually at least 5 bases. Where the mutation is an insertion or substitution, which exceeds about 20 bp, it is generally not necessary to include wild-type sequence in the probes/primers. For insertions or substitutions of fewer than 5 bp, preferred nucleic acid portions comprise and flank the substitution/insertion with at least 2, preferably at least 3, more preferably at least 4, most preferably at least 5 bases. For substitutions or insertions from about 5 to about 20 bp, it is usually necessary to include both the entire insertion/substitution and at least 2, usually at least 3, more usually at least 4, most usually at least 5 basis of wild-type sequence of at least one flank of the substitution/insertion.

The wording ‘stringent hybridization conditions’ is a term of art understood by those of ordinary skill in the art. For any given nucleic acid sequence, stringent hybridization conditions are those conditions of temperature, chaotrophic salts, pH and ionic strength which will permit hybridization of that nucleic acid sequence to its complementary sequence and not to substantially different sequences. The exact conditions, which constitute “stringent” conditions, depend upon the nature of the nucleic acid sequence, the length of the sequence, and the frequency of occurrence of subsets of that sequence within other non-identical sequences. By varying hybridization conditions from a level of stringency at which non-specific hybridization conditions occurs to a level at which only specific hybridization is observed, one of ordinary skill in the art can, without undue experimentation, can determine conditions which will allow a given sequence to hybridize only with complementary sequences. Hybridization conditions, depending upon the length and commonality of a sequence, may include temperatures of 20° C.-65° C. and ionic strengths from 5× to 0.1×SSC. Highly stringent hybridization conditions may include temperatures as low as 40-42° C. (when denaturants such as formamide are included) or up to 60-65° C. in ionic strengths as low as 0.1×SSC. These ranges, however, are only illustrative and, depending upon the nature of the target sequence, and possible future technological developments, may be more stringent than necessary.

In yet another embodiment the invention provides a method for the preparation of a diagnostic assay to detect the presence of a motor neuron disease, particularly distal HMN type II, in a human comprising detecting at least one mutation in the nucleotide sequence of SEQ ID NO: 1 in a tissue sample of said human, wherein said mutation results in a dominant mutation of HSP22 and wherein the presence of said mutation is indicative of a predisposition or the presence of a motor neuron disorder, particularly distal HMN II.

In yet another embodiment the invention provides a method for the preparation of a diagnostic assay to detect the presence of a motor neuron disorder, more particularly HMNII, in a human comprising detecting at least one mutation in the nucleotide sequence of SEQ ID NO: 1 in a tissue sample of said human, wherein said mutation is derived from Table 1 and wherein the presence of said mutation is indicative of a predisposition or the presence of a motor neuron disorder, more particularly HMNII.

When a diagnostic assay is to be based upon HSP22 proteins, a variety of approaches are possible. For example, diagnosis can be achieved by monitoring differences in the electrophoretic mobility of normal and mutant HSP22 protein. Such an approach will be particularly useful in identifying mutants in which charge substitutions are present, or in which insertions, deletions or substitutions have resulted in a significant change in the molecular mass of the resultant protein. Alternatively, diagnosis may be based upon differences in the proteolytic cleavage patterns of normal and mutant HSP22 protein, differences in molar ratios of the various amino acid residues, or by functional assays demonstrating altered function of the gene products. In some preferred embodiments, protein-based diagnostics will employ differences in the ability of antibodies to bind to normal and mutant HSP22 proteins. Such diagnostic tests may employ antibodies, which bind to the normal proteins but not to mutant proteins, or vice versa. In particular, an assay in which a plurality of monoclonal antibodies, each capable of binding to a mutant epitope, may be employed. The levels of anti-mutant examples binding in a sample obtained from a test subject (visualized by, for example, radiolabeling, ELISA or chemiluminescence) may be compared to the levels of binding to a control sample. Such antibody diagnostics may be used for in situ immunohistochemistry using biopsy samples of (CNS) tissues obtained antemortem or postmortem or may be used with fluid samples such a cerebrospinal fluid or with peripheral tissues such as white blood cells.

In yet another embodiment the invention provides a transgenic non-human animal comprising a vector comprising a dominant mutant of HSP22.

In yet another embodiment the invention provides a transgenic non-human animal comprising a vector comprising a mutation of HSP22 listed in Table 1.

Since the isolated HSP22 mutations are dominant (dominant negative), an alternative method for constructing a cell line is to engineer genetically a mutated gene, or a portion thereof into an established (either stably or transiently) cell line of choice. In another embodiment, the present invention provides a transgenic non-human animal that carries in its somatic and germ cells at least one integrated copy of a human DNA sequence that encodes a mutant HSP22 protein or fragment thereof. It is expected that the transgenic non-human animal, for example a transgenic mouse, will have a particular value because likewise in the human HMNII patients with the same pathogenic mutations in HSP22, a transgenic animal with a motor neuron disease is expected. In a preferred example it may be possible to excise the mutated HSP22 gene for use in the creation of transgenic animals containing the mutated gene. In another example, an entire human HSP22 mutant allele may be cloned and isolated, either in parts or as a whole, in a cloning vector (e.g. cosmid or yeast or human artificial chromosome). The human variant HSP22 mutant, either in parts or in whole, may be transferred to a host non-human animal, such as a mouse or a rat. As a result of the transfer, the resultant transgenic non-human animal will preferably express one or more mutant HSP22 polypeptides. Most preferably, a transgenic non-human animal of the invention will express one or more mutant HSP22 polypeptides in a motor neuron-specific manner (e.g. dorsal root ganglia). Alternatively, one may design minigenes encoding mutant HSP22 polypeptides. Such mini-genes may contain a cDNA sequence encoding a mutant HSP22 polypeptide, preferably full-length, a combination of HSP22 gene exons, or a combination thereof, linked to a downstream polyadenylation signal sequence and an upstream promoter (and preferably enhancer). Such a mini-gene construct will, when introduced into an appropriate transgenic host (e.g., mouse or rat), express an encoded mutant HSP22 polypeptide.

Another approach to create transgenic animals is to target a mutation to the desired gene by homologous recombination in an embryonic stem (ES) cell line in vitro followed by microinjection of the modified ES cell line into a host blastocyst and subsequent incubation in a foster mother (see Frohman and Martin (1989) Cell 56:145). Alternatively, the technique of microinjection of the mutated gene, or a portion thereof, into a one-cell embryo followed by incubation in a foster mother can be used. Various uses of transgenic animals are known in the art. Alternatively, site-directed mutagenesis and/or gene conversion can be used to mutate a murine (or other non-human) HSP22 gene allele, either endogenous or transfected. The procedure for generating transgenic rats is similar to that of mice (Hammer et al., Cell 63; 1099-112 (1990)). Thirty day-old female rats are given a subcutaneous injection of 20 IU of PMSG (0.1 cc) and 48 hours later each female placed with a proven male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBS (Dulbecco's phosphate buffered saline) with 0.5% BSA and the embryos collected. Cumulus cells surrounding the embryos are removed with hyaluronidase (1 mg/ml). The embryos are then washed and placed in EBSS (Earle's balanced salt solution) containing 0.5% BSA in a 37.5° C. incubator until the time of microinjection. Once the embryos are injected, the live embryos are moved to DPBS for transfer into foster mothers. The foster mothers are anesthetized with ketamine (40 mg/kg, ip) and xylazine (5 mg/kg, ip). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10-12 embryos are transferred into each rat oviduct through the infundibulum. The incision is then closed with sutures, and the foster mothers are housed singly.

Electrophysiological, behavioral, morphological and immunohistochemical investigations allow to compare the phenotype of the transgenic mice with the phenotype of distal HMN patients. Electrophysiological investigations are performed on transgenic mice and their wild type littermates. Nerve conduction velocities (NCV's) and amplitudes of the n. sciaticus and the n. tibialis are determined using needle electromyography (EMG) after anaesthesia, as described (Montag, D. et al (1994) Neuron, 13, 229-246). Sensory and motor impairments of the transgenic mice can be measured by using standardized clinical tests (SHIRPA protocol), acute thermal and mechano-sensory pain tests, measuring of general spontaneous activity, footprint analysis, grip strength measurements and rotarod test. Morphological examination of normal and transgenic animals enables to characterise specific changes particularly in nerve and muscle. For this purpose one can use light microscopy, transmission electron microscopy and immunohistochemistry. These models can be crossed with other animal models known for motor neuron disease, such as the SOD1 mouse for ALS. The models are relevant for screening pharmacological agents with the aim to develop therapeutic tools.

In another embodiment HSP22 can be used for the preparation of a medicament to treat a motor neuron disorder disease. In yet another embodiment HSP22 can be used for the preparation of a medicament to treat HMN II.

The term ‘medicament to treat’ relates to a composition comprising HSP22 and a pharmaceutically acceptable carrier or excipient (both terms can be used interchangeably) to treat diseases as indicated above. Generally, the medicament is administered so that the protein, polypeptide, peptide of the present invention is given at a dose between 0.5 ng/kg/day to 500 ng/kg/day, preferably in a dose range of 1 μg/kg/day and 10 mg/kg/day, more preferably between 10 μg/kg/day and 5 mg/kg/day, most preferably between 0.1 mg/kg/day and 2 mg/kg/day. Preferably, it is given as a bolus dose. Continuous infusion may also be used and includes continuous subcutaneous delivery via an osmotic minipump. A pharmaceutical composition of the invention is formulated to be compatible with its intended route or administration. Examples of routes of administration include parenteral, e.g., intravenous, intradermal, subcutaneous, oral (e.g. inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminium monostearate and gelatin. Sterile injectable solutions can be prepared by incorporating the active compound (e.g., chimeric peptide) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser, which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery. In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. In some instances, the reagent can be administered intrathecally or intracerebrally. Such an administration can for example be carried out by a surgical procedure implanting a catheter coupled to a pump device. The pump device can also be implanted or be extracorporally positioned.

Administration of the reagent can be in intermittent pulses or as a continuous infusion. Devices for injection to discrete areas of the brain are known in the art (see, e.g., U.S. Pat. Nos. 6,042,579; 5,832,932; and 4,692,147). Reagents containing compositions can be administered in any conventional form for administration of a protein. A reagent can be administered in any manner known in the art in which it may either pass through or by-pass the blood-brain barrier.

Methods are allowing factors to pass through the blood-brain barrier include minimizing the size of the factor, providing hydrophobic factors which may pass through more easily, conjugating the protein reagent or other agent to a carrier molecule that has a substantial permeability coefficient across the blood brain barrier (see, e.g., U.S. Pat. No. 5,670,477).

Reagents, derivatives, and co-administered agents can be incorporated into pharmaceutical compositions suitable for administration. Such compositions typically comprise the agent and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

Supplementary active compounds can also be incorporated into the compositions.

Modifications can be made to the agents to affect solubility or clearance of the peptide. In some cases, the composition can be co-administered with one or more solubilizing agents, preservatives, and permeation enhancing agents.

In another embodiment the present invention provides the nucleic acid of HSP22 for the transfection of cells in vitro and in vivo. In a preferred embodiment transfected cells are motor neurons. This nucleic acid can be inserted into any of a number of well-known vectors for the transfection of target cells and organisms as described below. The nucleic acids are transfected into cells, ex vivo or in vivo, through the interaction of the vector and the target cell.

The nucleic acid for HSP22, under the control of a promoter, then expresses HSP22, thereby mitigating the effects of absent, partial inactivation, or abnormal expression of HSP22. Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, Nabel & Feigner, TIBTECH 11:211-217 (1993); Mintani & Caskey, TIBTECH 11:162-166 (1993); Mulligan, Science 926-932 (1993); Dillon, TIBTECH 11:167-175 (1993); Van Brunt, Biotechnology 6(10): 1149-1154 (1998); Vigne, Restorative Neurology and Neuroscience 8:35-36 (1995); Kremer & Perricaudet, British Medical Bulletin 51(1); 31-44 (1995); Haddada et al., in Current Topics in Microbiology and Immunology (Doerfler & Böhm eds., 1995); and Yu et al., Gene Therapy 1:13-26 (1994)). Delivery of the gene or genetic material into the cell is the first critical step in gene therapy treatment of disease. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. Lipofection is described in, e.g., U.S. Pat. No. 5,049,386, U.S. Pat No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Flegner, WO 91/17424, WO 91/16024. Delivery can be to cells (ex vivo administration) or target tissues (in vivo administration). The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); U.S. Pat. Nos. 4,186,183, 4,217,344, 4,235,871, 4,261,975, 4,485,054, 4,501,728, 4,774,085, 4,837,028, and 4,946,787). The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long-term expression of the inserted transgene. Additionally, high transduction efficiencies have been observed in many different cell types and target tissues. The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised on cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); PCT/US94/05700. In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., U.S. Pat. No. 4,797,368; WO 93/24641; Kotin, Human Gene Therapy 5:793-801 (1994); Muzyczka. Construction of recombinant AAV vectors is described in a number of publications, including U.S. Pat. No. 5,173,414; Hermonat & Muzyczka, Proc. Natl. Acad. Sci. U.S.A. 81:6466-6470 (1984); and Samulski et al., J. Virol. 63:03822-3828 (1989). In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94/22 12133-12138 (1997)); Pa317/pLASN was the first therapeutic vector used in a gene therapy trials. (Blaese et al., Science 270:475-480 (1995)). Transduction efficiencies of 50% greater have been observed for MFG-S packaged vectors (Ellem et al., Immunol. Immunother. 44(1):10-20 (1997); Dranoff et al., Hum. Gene Ther. 1:111-2 (1997)). Recombinant adeno-associated virus vectors (rAAV) are a promising alternative gene delivery systems based on the defective and non-pathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet 351:9117 1702-3 (1998). Replication-deficient recombinant adenoviral vectors (Ad) are predominantly used transient expression gene therapy, because they can be produced at high titer and they readily infect a number of different cell types. Most adenovirus vectors are engineered such that a transgene replaced the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Sterman et al., Hum. Gene Ther. 9:7 1083-1089 (1998); Alvarez et al., Hum. Gene Ther. 5:597-613 (1997); Topf et al., Gene Ther. 5:507-513 (1998)). Packaging cells are used to form virus particles that are capable of infecting a host cell. Such cells include 293 cells, which package adenovirus, and ψ2 cells or PA317 cells, which package retrovirus.

Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest, For example, Han et al., Proc. Natl. Acad. Sci. U.S.A. 92/9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells. Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3^{rd} ed. 1994)) and the references cited therein for a discussion of how to isolate and culture cells from patients). In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176: 1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al., J. Exp. Med. 176:1693-1702 (1992)). Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Administration is by any of the routes normally used for introducing a molecule into ultimate contact with blood or tissue cells. The nucleic acids are administered in any suitable manner, preferably with pharmaceutically acceptable carriers. Suitable methods of administering such nucleic acids are available and well known to those of skill in the art, and, although more than one route can be used to administer a particular composition, a particular route can often provide a more immediate and more effective reaction than another route.

### EXAMPLE

1. A New Locus for HMNII

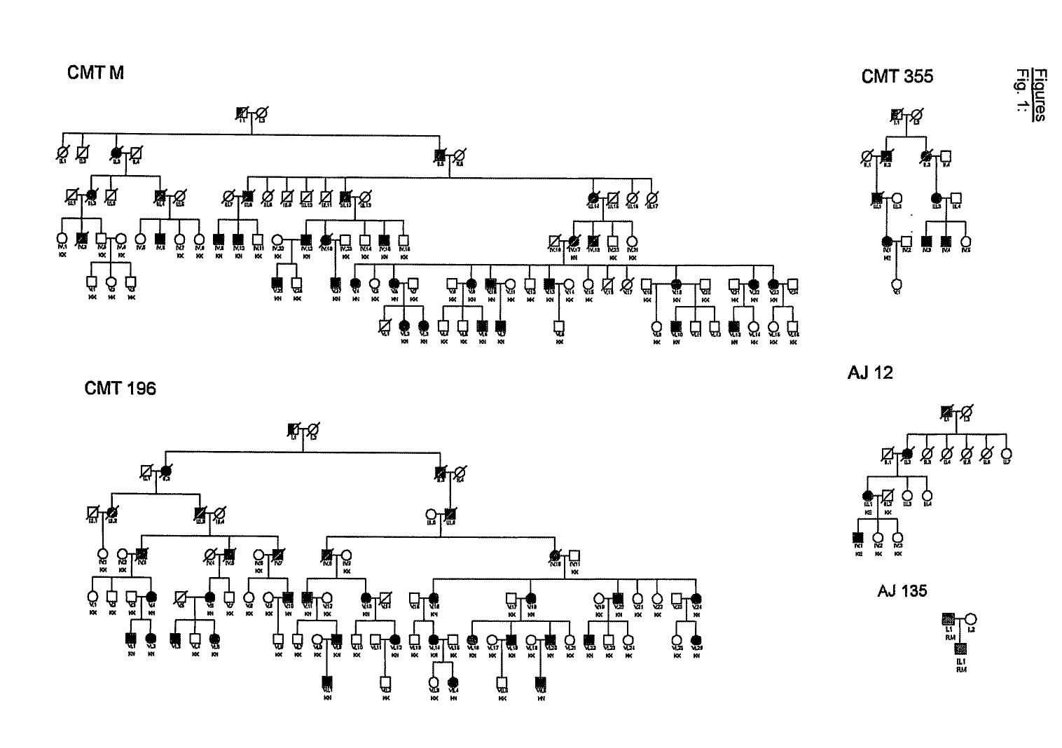

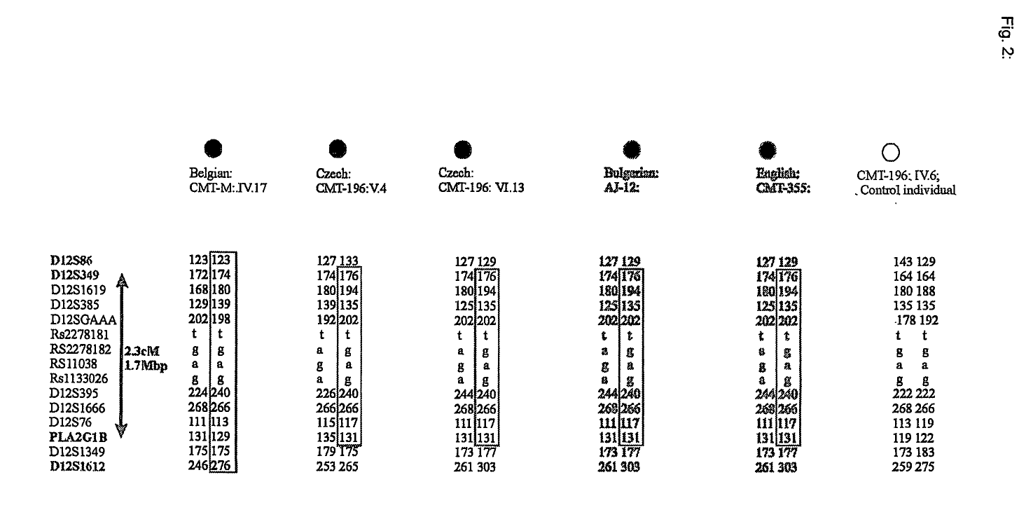

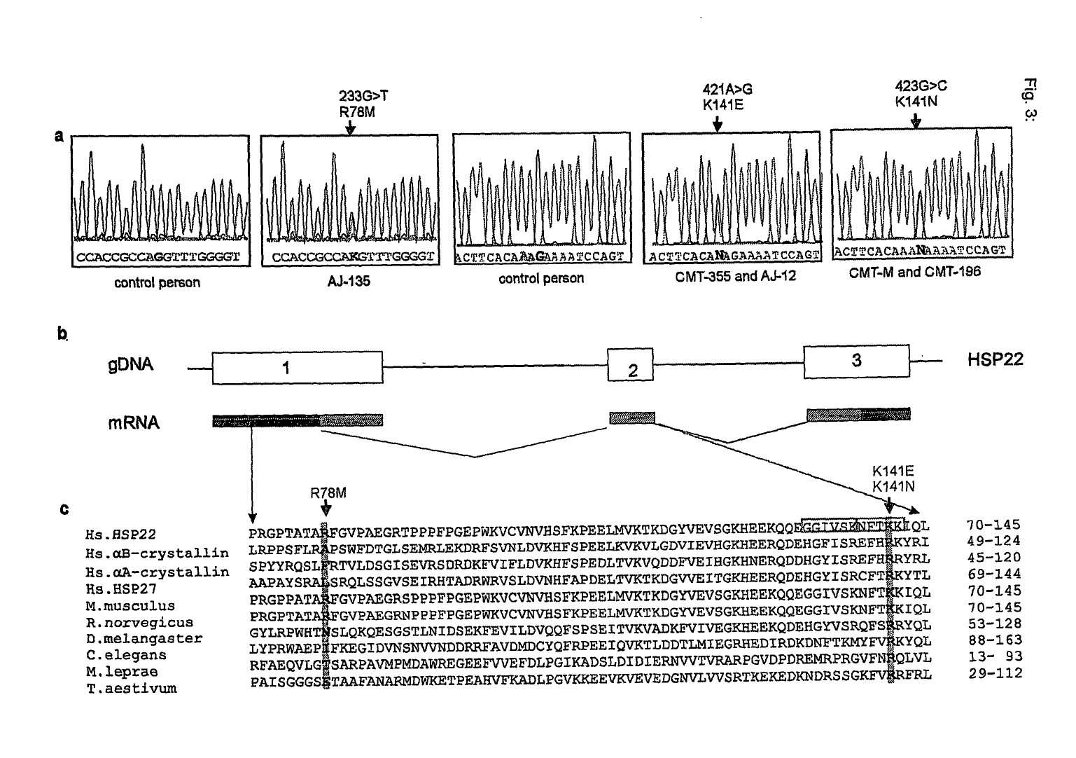

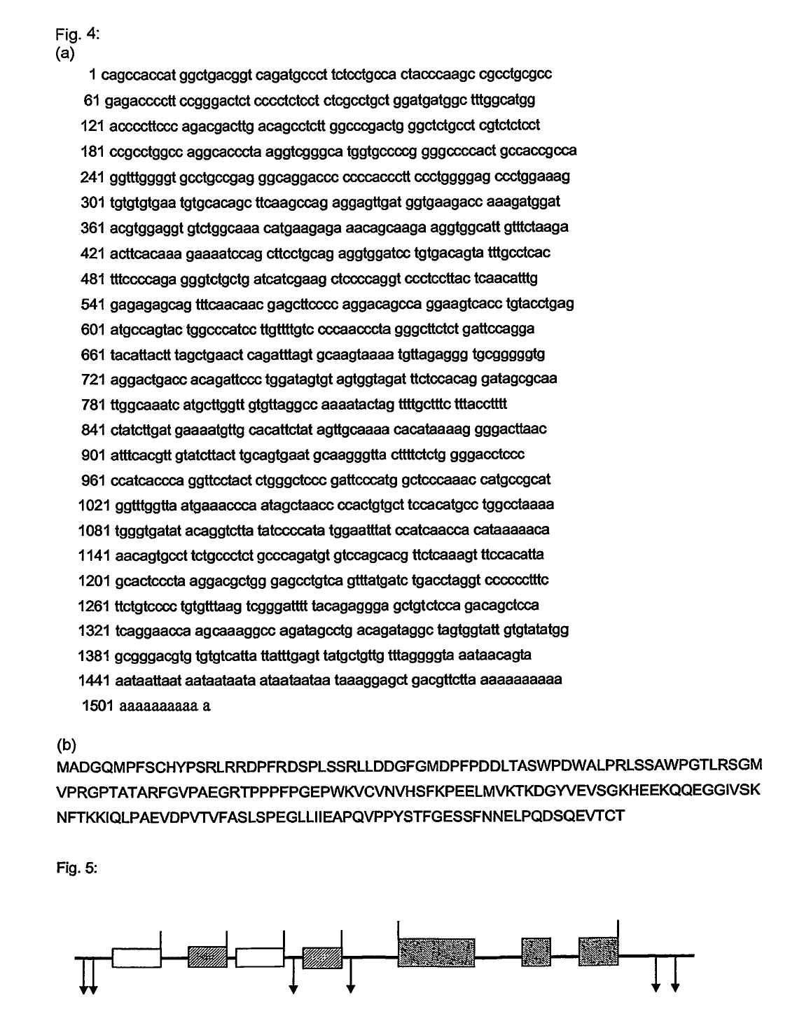

We previously mapped the distal HMN type II locus to a 5 Mb candidate region on chromosome 12q24.3 in a Belgian family⁵⁻⁷. In this study, we included three other families with autosomal dominant distal HMN; a Czech, Bulgarian and English family with 33, 4 and 2 affected individuals, respectively (FIG. 1). The phenotype in these families was strikingly similar to the previously reported Belgian family⁵ (FIG. 1). In the Belgian and Czech families we performed a haplotype analysis using short tandem repeat (STR) markers from the distal HMN II locus, and found several recombinants reducing the distal HMN II region to 1.7 Mb between D12S349 and PLA2G1B (FIG. 2). From this refined region, which contains nine known genes of which five genes had previously been excluded⁸⁻⁹, we selected four genes for mutation analysis: PRKAB1 (protein kinase, AMP-activated beta 1), CIT (citron rho-interacting, serine-threonine kinase), SIRT4 (sirtuin 4) and HSP22 (small stress protein or heat shock protein kinase synonyms: H11, HspB8, E2IG1). For each of these, we sequenced all known exons and intron-exon boundaries and did not find a disease-associated mutation in PRKAB1, CIT and SIRT4. However, in exon 2 of HSP22, encoding the central a crystallin domain (FIGS. 3b,c), we found a c.423G>C heterozygous transversion (K141N) in the Belgian and Czech families (FIG. 3a). A second c.421A>G heterozygous transition occurring on the same lysine residue (K141E) was found in the Bulgarian and English family (FIG. 3a). Both missense mutations showed perfect co-segregation with the distal HMN II phenotype in all 3 families and yielded a maximum two-point lod score of 9.4 and 9.2 between the mutation and disease in the Belgian and Czech families respectively. The Bulgarian and English families were too small to reach conclusive linkage. Both HSP22 mutations were absent in 400 control chromosomes. Since the Belgian and Czech distal HMN II families share the same HSP22 mutation, haplotype analysis with 11 STR markers and 4 intragenic HSP22 single nucleotide polymorphisms (SNPs) was performed and the results suggest that both families are likely distantly related (FIG. 2).

2. Expression Analysis

We performed an expression study using Northern blotting and RT-PCR and detected ubiquitous expression in human (2.0 kb) and mouse (1.8 kb), confirming the expression profile reported previously¹⁰ and in the UniGene Database (Hs.111676 and Mm.21549). Poly A⁺ RNA from different parts of human brain showed ubiquitous expression with the highest level detected in spinal cord. Furthermore, RT-PCR analysis of cDNA from mouse dorsal root ganglia and ventral horn at E13, showed expression of Hsp22 in sensory and motor neurons respectively.

3. Functional Analysis of HSP22 Mutations

The missense mutations K141N and K141E, identified in the distal HMN II families are located within the α-crystallin domain. Alignment of HSP22 orthologues shows that both mutations target a highly conserved positively charged lysine residue in the α-crystallin domain (FIG. 3c). This lysine residue also lies adjacent to a predicted N-myristoylation site, and within the putative protein kinase C (PKC) phosphorylation and N-glycosylation sites (FIG. 3c). These motifs are implicated in the proper protein folding, oligomerization, sorting and transport of proteins¹⁷. When glycosylation is inhibited, the most common observed effect is the generation of misfolded, aggregated proteins that fail to reach a functional state¹⁷. HSP22 was identified as a phosphoprotein that interacts with HSP27¹⁰ and that might be involved in modulation of HSP27 activities. Forced expression of HSP22 was recently shown to be regulated by DNA methylation and triggers apoptosis in human tumor cell lines.²² Since HSP22 is a known binding partner of HSP27, we investigated the effect of HSP22 K141N and K141E mutations on their interaction with HSP27 by co-immunoprecipitation experiments. To this end, EGFP-tagged HSP22 wild type (wt), K141N, and K141E mutants were transiently expressed in simian fibroblast cells (COS) and human embryonic kidney cells (HEK293T). Fusion proteins were immunoprecipitated using a polyclonal anti-EGFP antibody and immunoprecipitates were analyzed for the presence of HSP27. Endogenous HSP27 co-immunoprecipitated with EGFP-HSP22wt. Conversely, immunoprecipitation of EGFP-tagged HSP27 pulled down endogenous HSP22wt from COS cells or human neuronal cells (SHSY5Y). Immunoprecipitation of the HSP22 mutants from COS cells also revealed association with endogenous HSP27, indicating that the missense mutations do not destroy interaction between HSP22 and HSP27. Interestingly, both HSP22 mutants pulled down more HSP27 protein in comparison to HSP22wt. The latter was shown after careful quantitation of chemiluminescence detection (ECL) densitometry of obtained immunoprecipates from three independent experiments. To verify the specificity of the HSP22/HSP27 interaction, HEK293T that do not express HSP27, were co-transfected with EGFP-HSP22 (wt or mutants) and HSP27 containing V5 and HIS₆ epitope tags. Identical results were obtained as with COS cells: both HSP22wt and mutant HSP22 not only interact with HSP27, but both mutants pulled-down more HSP27 in comparison to HSP22wt. Intracellular desmin aggregates were observed in a familial type of desmin-related myopathy, which is associated with a missense mutation (R120G) in αβ-crystallin gene¹⁸⁻¹⁹. To test whether the K141N and K141E mutations can lead to the formation of HSP22 aggregates, we performed immunofluorescence experiments in which the expression of HSP22 was analyzed 48 hours after transfection of COS cells (FIG. 4). The COS cells expressing EGFP-HSP22wt showed homogeneous distribution throughout the cytoplasm and nucleus. In contrast, expression of K141N and K141E HSP22 mutants resulted in the presence of cytoplasmic and/or perinuclear HSP22-labelled aggregates. The percentage of aggregate-positive cells was significantly increased in K141N and K141E HSP22 transfected COS cells. (HSP22wt=2.9%±0.094; K141N=61.93%±1.96; K141E=59.13%±3.72). Data are means±SE of three independent experiments. Immunoblot analysis of the HSP22 wt and mutants in COS cells after 48 hours of transfection showed similar HSP22 expression levels in the supernatants but no expression was detected in the pellets. In conclusion, we demonstrated that mutations in HSP22 cause a motor neuron disorder or more specifically distal HMN II. We showed that the mutations in the HSP22 protein do not disrupt interaction with HSP27 but instead strengthen the interaction, and leads to the formation of aggregates. Therefore, HSP22 mutations may cause a ‘gain-of-function’ effect, which may lead to dysfunction of axonal transport and dysregulation of the cytoskeleton causing motor neuron death in distal HMN. However other important unknown mechanisms may be involved. In recent years, evidence has been obtained for the crucial role of some sHSPs in neuronal apoptosis and muscle function^{19,23}. A point mutation in the αβ-crystallin gene causes a severe desmin-related myopathy in humans¹⁹. HSPB2 associates specifically and activates the myotonic dystrophy protein kinase which causes myotonic dystrophy¹².

4. Overexpression of HSP22 in Neuronal Cells

We investigated if the overexpression of mutant HSP22 or mutant HSP27 resulted in induced cell death or in prevention of cell division in neuronal cells (e.g. N2A cells). After 48 hours, the MTS (cell viability assay by checking the integrity of the mitochondria) values obtained for the mutant HSP expressing N2a cells are lower in comparison to the N2a cells transfected with wild type HSP constructs (both HSP27 and HSP22). The transfection efficiencies were approximately 90%. As the N2a cells are very small and have almost no cytoplasm around the nucleus it is difficult (using conventional fluorescence microscopy) to find perinuclear aggregates. We found that N2a cells transfected with mutant HSP22/HSP27 contained multinucleated cells.

5. Detection of HSP22 in an Animal Model of Motor Neuron Disease

On Northern blots of total spinal cord of G93A mutant SOD1 transgenic mice (ALS mouse model) at different ages (80 days=pre-symptomatic, 120 days=early symptomatic, and 150 days=late symptomatic), we found an over-expression of wild type HSP22. The hybridization signals were normalized with a normal mouse spinal cord of 80 days, and to the signal of actin as probe. Especially, at the end-stage of the disease, HSP22 is over-expressed compared to the normal mouse.

6. Isolation of Motor Neurons from E13 Mouse

Dissection of E13 mouse embryos can be carried out in HBSS (Hanks' Balanced Salt Solution, Life Technologies, San Diego, Calif.). To obtain the ventral horn (VH), spinal cords are stripped of meninges and dorsal root ganglion (DRG), and the dorsal half of the cord is removed (Juurlink B. H. J. (2003) in: Protocols for neuronal cell culture, S. Fedoroff and A. Richardson, eds. The Human Press Inc. pp. 39-51). Isolation of DRG is carried out by a “pull-up” technique as previously described (Johnson M. I. and Bunge R. P. (1992) in: Protocols for neuronal cell culture, S. Fedoroff and A. Richardson, eds. The human Press inc. pp. 13-38). A single cell suspension can be made for the VH (Camu W. and Henderson C. E. (1992) J. Neurosci. Methods 44, 59-70). Small pieces of VH (1 mm) are digested for 15 minutes in 0.05% trypsin (ICN, Irvine, Calif.) in HBSS at 37° C. After treatment with DNase I containing suspension medium (L15 with 3.6 mg/ml glucose, 20 nM progesterone, 5 μg/ml insulin, 0.1 mM putrescin, 0.1 mg/ml conalbumin, 30 nM sodium selenite, 100 IU/ml penicillin, 100 μg/ml streptomycin, 2% horse serum, 1× Glutamax I and 0.1 mg DNase I), the tissue is mechanically dissociated by trituration with 1 ml suspension medium enriched with 0.4% BSA and 20 μg DNase I. TrypanBlue is used to count all viable cells in a Counting Chamber. The cell suspension obtained from the VH is layered on a 6.4% metrizamide (Sigma) cushion with two spinal cord equivalents per tube and after centrifugation a motor neuron-enriched cell population was obtained from the top of the metrizamide cushion. The isolated motor neurons are purified on a 4% BSA cushion, suspended in suspension medium and cell counting was performed using TrypanBlue (Schnaar R. I. and Schaffner A. E. (1981) J. Neurosci. 1, 204-217). The quality of the motor neuron cell suspensions is analyzed by immunocytochemistry before and after the metrizamide gradient isolation procedure. NeuN (Neuronal Nuclei), a mouse IgG monoclonal antibody strongly staining the nucleus and weakly staining the cytoplasm of postmitotic neurons, is used as a neuron-specific marker. Nuclear counterstaining is performed with haematoxylin.

7. SNP Map of HSP22

To investigate whether genetic variations in HSP22 have a modulating effect on the disease process of ALS we have built an SNP map of HSP22 on the basis of SNPs identified through mutation analysis (in combination with the novoSNP software, http://www.molgen.ua.ac.be/bioinfo/) in 24 ad random selected sporadic ALS patients. For HSP22 we detected 6 different SNPs (FIG. 5). With the help of Arlequin (http://lgb.unige.ch/arlequin/), which estimates the most frequent haplotypes, we selected three SNPs (−891A>T unpublished SNP, 644A>G published SNP Rs.11038, 684G>A published SNP Rs.1133026), that have a minor allele frequency>5%.

8. Generation of Murine Transgenic Lines for HSP22

We generated constructs for transgenic mice to model distal HMN by overexpression of human HSP22 mutant K141N under the regulation of the chicken β-actin/rabbit β-globin hybrid (AG) promotor. The reason for choosing these mutants is that these are located in the α-crystallin domain of the small HSP proteins and the fact that our patients with these mutations have been well documented by clinical and electrophysiological studies.

For design of the transgenic construct, we cloned human HSP22 and a HA-tag in the pCAGGS vector. This vector contains the promoter region, regulatory elements and the 3′-UTR of the gene coding chicken β-actin/rabbit β-globin. This vector was kindly provided by Dr. C. Libert (Department of Molecular Biomedical Research, VIB) and proven to be successful for transgenic experiments. Linearised constructs for HSP22 mutant and wild type, devoid of all plasmid sequences are purified and microinjected into 1.5 days old pre-nuclear embryos. The background for transgenesis is C57BL6×CBA. The offspring is genotyped by PCR with human HSP22 cDNA primers and Southern blotting of tail-biopt DNA. Breeding founder animals to the F1 generation result into the establishment of stable transgenic lines.

Materials and Methods

Patients

Our study included four distal HMN families. In the Belgian family (CMT-M), we previously linked the distal HMN type II locus on chromosome 12q24.3⁵⁻⁶. The Czech (CMT-196), English (CMT-355) and Bulgarian (AJ-12) families were recently ascertained. From family members and control persons we isolated genomic DNA from total blood samples using a standard extraction protocol. Informed consent was obtained from all family members and the study was approved by the Institutional Review Board at the Universities of Antwerp, Sofia and Prague.

Genotyping and Genetic Linkage Analysis

PCR amplification was performed with dye-labeled STR primers on a DYAD thermocycler (MJ Research Inc, Waltham, USA). Fragment analysis was performed on an ABI3700 DNA sequencer and analyzed with the ABI GENESCAN 3.1 and GENOTYPER 2.1 software (Perkin-Elmer, Applied Biosystems Inc.). For linkage to the HSP22 gene and STR markers, two-point LOD scores were computed using the MLINK program of the FASTLINK package²⁷. We assumed autosomal dominant inheritance, a disease frequency of 1/10.000, equal male/female recombination rates, and a complete penetrance. LOD scores were calculated with equal allele frequencies assumed for all markers. The order of markers on the genetic and physical maps was established by consulting the genome database: http://www.gdb.org/ and http://www.ncbi.nlm.nih.gov/LocusLink/

Mutation Analysis

We used the NCBI Entrez Genome Map Viewer (http://www.ncbi.nlm.nih.gov/mapview/map_search.cgi?chr=hum_chr.inf&query), Ensembl Human Genome Server (http://www.ensembl.org/) and Genbank database (http://www.ncbi.nlm.nih.gov/entrez/query.fcgi?db=Nucleotide) to find known genes, ESTs and putative novel genes in the distal HMN II region. We determined the exon-intron boundaries of the candidate sequences by BLAST searches against the high throughput genome sequences (HTGS, NT_{—}009775). All exons of the PRKAB1 (NM_{—}006253), CIT (NM_{—}007174), SIRT4 (NM_{—}012240), and HSP22 (NM_{—}014365) genes were PCR-amplified using intronic primers (sequences available on request, Table 2, supplementary data). PCR products were sequenced using the DYEnamic ET Terminator Cycle Sequencing Kit (Amersham Pharmacia Biotech). The sequence reactions were loaded on the ABI3700 sequencer (Perkin-Elmer, Applied Biosystems Inc.). The data were collected and analyzed using the ABI DNA sequencing analysis software, version 3.6.

Expression Analysis

A plasmid clone IRALp962K0712, containing the complete human HSP22 cDNA sequences were obtained from RZPD (The Resource Center of the German Human Genome Project at http://www.rzpd.de/). T3- and T7-primers were used to make a HSP22 cDNA probe of 800 bp. This probe was used to hybridize the Human 12 and 8-lane Multiple Tissue and Brain Northern blot (Clontech). Total RNA was extracted from mouse muscle (NMRI, Navy Medical Research Institute, USA) using the Totally RNA Kit (Ambion). RT-PCR was carried out using the Random Primer DNA Labeling System (Life Technologies). The full-length mouse Hsp22 cDNA was used as a probe to hybridize the Mouse Multiple Tissues and Embryo's Northern blot (Clontech). Northern blots were also hybridized with a β-actin cDNA probe (Clontech) as a control for RNA loading. Motor and sensory neurons were isolated from 13 days old mice embryos. Total RNA was extracted using the Totally RNA Kit (Ambion) and RT-PCR was carried out using the SMART PCR cDNA Amplification kit (Clontech). Mouse Hsp22 cDNA primers (musH11-F=‘5-ACCTTTGGGTAGGTGGCTCT-3’ and musH11-R=‘5-GGGATGGGAGCGAAGAAG-3’) were used to amplify Hsp22 cDNA fragment of 687 bp.

cDNA Cloning and Mutagenesis

The cDNA encoding full-length wild type human HSP22 or HSP27 (refs 10, 28)^{10,28} was cloned as a HindIII fragment into the pEGFP-C1 (Clontech) and pcDNA3.1V5/His TOPO (Invitrogen) vectors by PCR using the RZPD plasmids IRALp962K0712 (HSP22) and IRALp962H201 (HSP27) as templates. For site-directed mutagenesis of the HSP22 mutations c.423C>G and c.421A>G, we used the QuikChange site-directed mutagenesis kit (Stratagene). All constructs were verified by direct DNA sequencing.

Immunoprecipitation.

Cells were washed twice with ice-cold PBS (2.7 mM KCL, 1.47 mM KH₂PO₄, 137 mM NaCl, 8.1 mM Na₂HPO₄, pH 7.4) and lysed in PBS with 0.5% NP-40, 5 mM Levamisole, 10 mM Sodium fluoride, 8 mm Sodium-β-glycerophosphate and a protease inhibitor cocktail mix (Roche Diagnostics, Mannheim, Germany). Cells were disrupted by sonication and the crude extract was centrifuged at 4° C. for 10 min (14000 rpm). One milligram of proteins was incubated overnight with affinity purified anti-EGFP antibodies and subsequently incubated for 4 h with protein G-Sepharose (Amersham Pharmacia Biotech). The beads were washed five times with the lysis buffer, boiled for 5 min in Laemmli sample buffer and proteins were fractionated by SDS-PAGE followed by Western blotting. Proteins were visualized by enhanced chemiluminescence detection (ECL kit; Amersham Pharmacia Biotech).

Immunostaining and Microscopy

The cells were viewed directly for EGFP fluorescence after fixation with 3% paraformaldehyde in phosphate-buffered saline for 20 min at room temperature. Cells were examined using a Zeiss Axioplan II epifluorescence microscope equipped with a ×40 objective. Images were captured using a cooled CCD Axiocam Camera and KS100 software (Zeiss, Gottingen, Germany).

Miscellaneous

Affinity purification of anti-EGFP antibodies was performed according to standard procedures (Gettemans et al., 1995). Protein concentrations were determined by the method of Bradford (1976) using bovine serum albumin as a standard. SDS-PAGE was carried out according to Matsudaira and Burgess (1978).

Genbank Accession Numbers

Protein sequences: human HSP22, NP_{—}055180; mouse Hsp22, NP_{—}109629; rat HSP22, NP_{—}446064; Drosophila melanogaster HSP22, NP_{—}523996; Caenorhabditis elegans HSP22, NP_{—}509045; Triticum aestivum (bread wheat), GI 17942916, Mycobacterium leprae Hsp16.7, P12809. Genomic sequences: (NT_{—}009775). PRKAB1 (NM_{—}006253), CIT (NM_{—}007174), SIRT4 (NM_{—}012240), and HSP22 (NM_{—}014365). HSP22 SNPs: Rs2278181, RS2278182, RS11038, RS113302.

Tables

TABLE 1
Mutations which were identified in the HSP22 protein, the numbering
corresponds with the nucleotide numbering in SEQ ID NO: 1
(first column) and amino acid numbering in
SEQ ID NO: 2 (second column).

| | |
|---|---|
| c.423G > C | p.K141N |
| c.421A > G | p.K141E |
| c.233G > T | p.R78M |

TABLE 2
Primer sequences for sequencing of candidate genes

| | | | Primer position | |
|---|---|---|---|---|
| | | | according | PCR- |
| Gene and | Forward primer | Reverse primer | to the first | product |
| primer name | sequences (5′ −−> 3′) | sequences (5′ −−> 3′) | ATG (start-end) | (bp) |
| PRKAb1ex1 | GGTTGGAAAGTGTCGGTTT | GGAGGGTTCCTCTCCTCAAC | 1-435 | 481 |
| PRKAB1ex2 | TCCGATCCTAACCATGAACC | TTTTCCACTAGGCATCCATTTT | 436-598 | 397 |
| PRKAB1ex3-4 | TCTGTAGCTGGTTTGGCAAG | AGACTGTACAGCCCCCACCT | 599-807 | 621 |
| PRKAB1ex5 | CTTGGAACCAGTGCATCCTT | TTTGCAAGAGGTGGACACAG | 808-941 | 360 |
| PRKAB1ex6-7 | GGGGAGAATCTTGGTTTCCA | ACCAGGGCAGGTATGAAATG | 942-1088 | 585 |
| SIRT4ex1 | TGGTGATCAAGACAGCCAAG | CTGGGCAACAGAGGGAGACT | 1-518 | 691 |
| SIRT4ex2 | CGTCTCTGACAGCTTTGTGC | CTGCACGGAGAAAAGACACA | 519-816 | 492 |
| SIRT4ex3 | TTGGGAGTCCTGGAGAGACA | AGTATGACCCCTGTGCAAGA | 817-965 | 500 |
| CITex1 | GTTGGAACCCTTGGGAGAACGTGTCACGTGGG | GTGTCACGTGGGTTCAGAAA | 255-452 | 382 |
| | TTCAGAAA | | | |
| CITex2 | TTTAGCACCAGGAGGCTTGTCCCGACCAAAGT | CCCGACCAAAGTAATCTCCA | 453-647 | 398 |
| | AATCTCCA | | | |
| CITex3 | AACCATGGGACATTTTTGGA | AGAGACGGACCAGCCTTCTT | 648-786 | 354 |
| CITex4 | GACACTGTGGGAGGAGGAGATCTTTCTCCGTG | TCTTTCTCCGTGAAGGTTCG | 787-887 | 351 |
| | AAGGTTCG | | | |
| CITex5 | CGAAGTGCTGGGATTACAGGGGTGCCATGCCT | GGTGCCATGCCTGAAATTAG | 888-1030 | 321 |
| | GAAATTAG | | | |
| CITex6 | GTCCACTGAGCCATGAATGATGACTCAGTACT | TGACTCAGTACTGTTTGTGTTGGA | 1031-1138 | 306 |
| | GTTTGTGTTGGA | | | |
| CITex7 | ACATCAACTTGGCAATGCACGCTTTTGTGGTT | GCTTTTGTGGTTTGCTCCTC | 1139-1254 | 434 |
| | TGCTCCTC | | | |
| CITex8 | CTTGAGCTCCCAACTTCAGGCTGTGCATTGCC | CTGTGCATTGCCAAGTTGAT | 1255-1419 | 489 |
| | AAGTTGAT | | | |
| CITex9 | CAGCTTCAACAGGGGAAAAATTCCCTTTCTGT | TTCCCTTTCTGTGGGTTGTC | 1420-1557 | |
| | GGGTTGTC | | | |
| CITex10-11 | ACTGGGGAGACCTGGGTTAGAGAGGAAGGGAA | AGAGGAAGGGAAGGGTCCAG | 1558-1830 | 568 |
| | GGGTCCAG | | | |
| CITex12-13 | AGCCTGAGGGGAATCAAAATTCCCCTTGTTCT | TCCCCTTGTTCTTGTTCCTG | 1831-2105 | 684 |
| | TGTTCCTG | | | |
| CITex14-15 | CATGAAACGTGGCTTCAACAGGTTTCTCTGGA | GGTTTCTCTGGATGGTTTGG | 2106-2323 | 520 |
| | TGGTTTGG | | | |
| CITex16 | ACGAGCTCTGTGGGAAGAGATCTGTGTGGCCT | TCTGTGTGGCCTCTTGTGAC | 2324-2462 | 323 |
| | CTTGTGAC | | | |
| CITex17 | CAGTGCACTTTCCACACTGGTTCCTAGTTTTG | TTCCTAGTTTTGCCCCACAG | 2463-2556 | 242 |
| | CCCCACAG | | | |
| CITex18-19 | TCCATGTACCCTCCTCCAACAGAACAGCTGTG | AGAACAGCTGTGGACCTTGG | 2557-2793 | 486 |
| | GACCTTGG | | | |
| CITex20 | CGGATGCAATTCTTTTCCAGTGCTCCTCATTC | TGCTCCTCATTCTTCCATCA | 2794-2874 | 237 |
| | TTCCATCA | | | |
| CITex21-22 | TGGTTTAGTATCACTTCCTTCTGCCTTTGATT | TTTGATTTTCCCTCTTTTCACC | 2875-3133 | 535 |
| | TTCCCTCTTTTCACC | | | |
| CITex23 | TCAGTTCCCCAAGTCACTCCGTCAAGGAGGGG | GTCAAGGAGGGGGTTGCT | 3134-3439 | 455 |
| | GTTGCT | | | |
| CITex24 | TGATGATGTGGTCGAGCTAAA | GTGAGCACAGCAACTTCTGG | 3440-3461 | 598 |
| HSP22ex1 | CAGGGCTGAGGGCTACATC | GAGAGGCCGGCTGAACTT | 1-891 | 950 |
| HSP22ex2 | AGGGAGAGACCCCAGATCAT | TCATAGCCAGCCTTGGAAGT | 892-955 | 350 |
| HSP22ex3 | CCAACATTGTATGTCCCCAAACCCGCACCCTC | CCCGCACCCTCTAACATTT | 956-1114 | 450 |
| | TAACATTT | | | |

### REFERENCES

1. Harding, A. E. & Thomas, P. K. Hereditary distal spinal muscular atrophy. A report on 34 cases and a review of the literature. J. Neurol. Sci. 45, 337-348 (1980).
2. Puls, I. et al. Mutant dynactin in motor neuron disease. Nat. Genet. 33 (4), 455-456 (2003).
3. Grohmann, K. et al. Mutations in the gene encoding immunoglobulin μ-binding protein 2 cause spinal muscular atrophy with respiratory distress type 1. Nat. Genet. 29, 75-77 (2001).
4. Antonellis, A. et al. Glycyl tRNA Synthetase Mutations in Charcot-Marie-Tooth Disease Type 2D and Distal Spinal Muscular Atrophy Type V. Am. J. Hum. Genet. 72, 1293-1299 (2003).
5. Timmerman, V. et al. Linkage of distal hereditary motor neuropathy type II (distal HMN II) in a single pedigree. J. Neurol. Sci. 109, 41-48 (1992).
6. Timmerman, V. et al. Distal hereditary motor neuropathy type II (distal HMN II): Mapping of a locus to chromosome 12q24. Hum. Mol. Genet. 5, 1065-1069 (1996).
7. Irobi, J. et al. A clone contig of 12q24.3 encompassing the distal hereditary motor neuropathy type II gene. Genomics 65, 34-43 (2000).
8. Irobi, J. et al. Exclusion of 5 functional candidate genes for distal hereditary motor neuropathy type II (distal HMN II) linked to 12q24.3. Ann. Hum. Genet. 65, 517-529 (2001).
9. Irobi, J. et al. Mutation analysis of 12 functional candidate genes for distal hereditary motor neuropathy type II (distal HMN II) linked to 12q24.3. J. Peripheral Nervous System 7, 87-95 (2002).
10. Benndorf, R. et al. HSP22, a new member of the small heat shock protein superfamily, interacts with mimic of phosphorylated HSP27 ((3D)HSP27). Journal of Biological Chemistry 276, 26753-26761 (2001).
11. Boelens, W. C., Van Boekel, M. A & De Jong, W. W. HspB3, the most deviating of the six known human small heat shock proteins. Biochim. Biophys. Acta 1388, 513-516 (1998).
12. Suzuki, A. et al. MKBP, a novel member of the small heat shock protein family, binds and activates the myotonic dystrophy protein kinase. J. Cell Biol. 140, 1113-1124 (1998).
13. Arrigo A P & Landry J. The biology of the heat shock proteins and molecular chaperones. Cold Spring Harbor Laboratory, Cold Spring Harbor, N.Y., 335-373. 1994. Ref Type: Generic
14. Concannon, C. G., Gorman, A. M. & Samali, A. On the role of Hsp27 in regulating apoptosis. Apoptosis. 8, 61-70 (2003).
15. Bova, M. P., Mchaourab, H. S., Han, Y. & Fung, B. K. Subunit exchange of small heat shock proteins. Analysis of oligomer formation of alphaA-crystallin and Hsp27 by fluorescence resonance energy transfer and site-directed truncations. J. Biol. Chem. 275, 1035-1042 (2000).
16. Gusev, N. B., Bogatcheva, N. V. & Marston, S. B. Structure and properties of small heat shock proteins (sHsp) and their interaction with cytoskeleton proteins. Biochemistry-Moscow 67, 511-519 (2002).
17. Helenius, A. How N-linked oligosaccharides affect glycoprotein folding in the endoplasmic reticulum. Mol. Biol. Cell 5, 253-265 (1994).
18. Zobel, A. T. C. et al. Distinct chaperone mechanisms can delay the formation of aggresomes by the myopathy-causing R120G alpha B-crystallin mutant. Hum. Mol. Genet. 12, 1609-1620 (2003).
19. Vicart, P. et al. A missense mutation in the alphaB-crystallin chaperone gene causes a desmin-related myopathy. Nat. Genet. 20, 92-95 (1998).
20. Smith, C. C., Nelson, J., Aurelian, L., Gober, M. & Goswami, B. B. Ras-GAP binding and phosphorylation by herpes simplex virus type 2 RR1 PK (ICP10) and activation of the Ras/MEK/MAPK mitogenic pathway are required for timely onset of virus growth. J. Virol. 74, 10417-10429 (2000).
21. Perkins, D., Pereira, E. F., Gober, M., Yarowsky, P. J. & Aurelian, L. The herpes simplex virus type 2 R1 protein kinase (ICP10 PK) blocks apoptosis in hippocampal neurons, involving activation of the MEK/MAPK survival pathway. J. Virol. 76, 1435-1449 (2002).
22. Gober, M. D., Smith, C. C., Ueda, K., Toretsky, J. A. & Aurelian, L. Forced expression of the h11 heat shock protein can be regulated by DNA methylation and trigger apoptosis in human cells. J. Biol. Chem. 278, 37600-37609 (2003).
23. Benn, S. C. et al. Hsp27 upregulation and phosphorylation is required for injured sensory and motor neuron survival. Neuron 36, 45-56 (2002).
24. Vleminckx, V. et al. Upregulation of HSP27 in a transgenic model of ALS. J. Neuropathol. Exp. Neurol. 61, 968-974 (2002).
25. Kobayashi, Y., Kume, A, Ohtsuka, K. & Sobue, G. Chaperones, Hsp70 and Hsp40, suppress aggregate formation and apoptosis in cultured neuronal cells expressing truncated androgen receptor protein with expanded polyglutamine tract. Neurology 54, A359 (2000).
26. Adachi, H. et al. Heat shock protein 70 chaperone overexpression ameliorates phenotypes of the spinal and bulbar muscular atrophy transgenic mouse model by reducing nuclear-localized mutant androgen receptor protein. J. Neurosci. 23, 2203-2211 (2003).
27. Cottingham, R. W., Idury, R. M. & Schäffer, A. A. Faster sequential genetic linkage computations. Am. J. Hum. Genet. 53, 252-263 (1993).
28. Carper, S. W., Rocheleau, T. A. & Storm, F. K. Cdna Sequence of A Human Heat-Shock Protein Hsp27. Nucleic Acids Research 18, 6457 (1990).
29. Towbin, H., Staehelin, T. & Gordon, J. Electrophoretic transfer of proteins from polyacrylamide gels to nitrocellulose sheets: procedure and some applications. Proc. Natl. Acad. Sci. U.S.A 76, 4350-4354 (1979).
30. T'Jampens, D., Devriendt, L., De Corte, V., Vandekerckhove, J. & Gettemans, J. Selected BTB/POZ-kelch proteins bind ATP. Febs Letters 516, 20-26 (2002).

1. An isolated nucleic acid coding for a dominant negative, mutant HSP22 polypeptide, said nucleic acid containing in comparison to the wild type HSP22 encoding sequence set forth in SEQ ID NO: 1 one or more mutations wherein the presence of said nucleic acid is indicative for the presence of a motor neuron disorder.

2. An isolated nucleic acid according to claim 1 wherein the presence of said nucleic acid is indicative for distal hereditary motor neuropathy type II.

3. An isolated nucleic acid according to claim 1 wherein said mutations of HSP22 are set forth in Table 1.

4. A nucleic acid probe, which is a fragment of the nucleic acid sequences according to claim 1.

5. A recombinant vector comprising an isolated nucleic acid according to claim 1.

6. A host cell comprising a recombinant vector according to claim 5.

7. A method for the preparation of a diagnostic assay to detect the presence of a motor neuron disorder in a human comprising detecting at least one mutation in the nucleotide position of SEQ ID NO: 1 in a tissue sample of said human, wherein said mutation respectively results in a dominant mutation of HSP22 and wherein the presence of said mutation is indicative of the presence of a motor neuron disorder.

8. A method according to claim 7 wherein said mutations for HSP22 are set forth in Table 1.

9. A transgenic non-human animal comprising a vector according to claim 5.

10. Use of HSP22 for the manufacture of medicament for the treatment of a motor neuron disorder.

11. Use of HSP22 according to claim 10 wherein said motor neuron disorder is hereditary motor neuropathy type II.


SEQUENCE LISTING (79 sequences)

SEQ ID NO: 1 - DNA, 1511 bp, Homo sapiens
cagccaccatggctgacggtcagatgcccttctcctgccactacccaagccgcctgcgccgagaccccttccgggactctcccctctcctctcgcctgctggatgatggctttggcatggaccccttcccagacgacttgacagcctcttggcccgactgggctctgcctcgtctctcctccgcctggccaggcaccctaaggtcgggcatggtgccccggggccccactgccaccgccaggtttggggtgcctgccgagggcaggacccccccacccttccctggggagccctggaaagtgtgtgtgaatgtgcacagcttcaagccagaggagttgatggtgaagaccaaagatggatacgtggaggtgtctggcaaacatgaagagaaacagcaagaaggtggcattgtttctaagaacttcacaaagaaaatccagcttcctgcagaggtggatcctgtgacagtatttgcctcactttccccagagggtctgctgatcatcgaagctccccaggtccctccttactcaacatttggagagagcagtttcaacaacgagcttccccaggacagccaggaagtcacctgtacctgagatgccagtactggcccatccttgttttgtccccaaccctagggcttctctgattccaggatacattactttagctgaactcagatttagtgcaagtaaaatgttagagggtgcgggggtgaggactgaccacagattccctggatagtgtagtggtagatttctccacaggatagcgcaattggcaaatcatgcttggttgtgttaggccaaaatactagttttgctttctttaccttttctatcttgatgaaaatgttgcacattctatagttgcaaaacacataaaaggggacttaacatttcacgttgtatcttacttgcagtgaatgcaagggttacttttctctggggacctcccccatcacccaggttcctactctgggctcccgattcccatggctcccaaaccatgccgcatggtttggttaatgaaacccaatagctaaccccactgtgcttccacatgcctggcctaaaatgggtgatatacaggtcttatatccccatatggaatttatccatcaaccacataaaaacaaacagtgccttctgccctctgcccagatgtgtccagcacgttctcaaagtttccacattagcactccctaaggacgctgggagcctgtcagtttatgatctgacctaggtcccccctttcttctgtcccctgtgtttaagtcgggatttttacagagggagctgtctccagacagctccatcaggaaccaagcaaaggccagatagcctgacagataggctagtggtattgtgtatatgggcgggacgtgtgtgtcattattatttgagttatgctgttgtttaggggtaaataacagtaaataattaataataataataataataataataaaggagctgacgttcttaaaaaaaaaaaaaaaaaaaaaa

SEQ ID NO: 2 - protein, 196 aa, Homo sapiens
MADGQMPFSCHYPSRLRRDPFRDSPLSSRLLDDGFGMDPFPDDLTASWPDWALPRLSSAWPGTLRSGMVPRGPTATARFGVPAEGRTPPPFPGEPWKVCVNVHSFKPEELMVKTKDGYVEVSGKHEEKQQEGGIVSKNFTKKIQLPAEVDPVTVFASLSPEGLLIIEAPQVPPYSTFGESSFNNELPQDSQEVTCT

SEQ ID NO: 3 - protein, 76 aa, Triticum aestivum
PAISGGGSETAAFANARMDWKETPEAHVFKADLPGVKKEEVKVEVEDGNVLVVSRTKEKEDKNDRSSGKFVRRFRL

SEQ ID NO: 4 - protein, 76 aa, Drosophila melanogaster
GYLRPWHTNSLQKQESGSTLNIDSEKFEVILDVQQFSPSEITVKVADKFVIVEGKHEEKQDEHGYVSRQFSRRYQL

SEQ ID NO: 5 - protein, 76 aa, Homo sapiens
SPYYRQSLFRTVLDSGISEVRSDRDKFVIFLDVKHFSPEDLTVKVQDDFVEIHGKHNERQDDHGYISREFHRRYRL

SEQ ID NO: 6 - protein, 76 aa, Homo sapiens
LRPPSFLRAPSWFDTGLSEMRLEKDRFSVNLDVKHFSPEELKVKVLGDVIEVHGKHEERQDEHGFISREFHRKYRI

SEQ ID NO: 7 - protein, 76 aa, Homo sapiens
PRGPTATARFGVPAEGRTPPPFPGEPWKVCVNVHSFKPEELMVKTKDGYVEVSGKHEEKQQEGGIVSKNFTKKIQL

SEQ ID NO: 8 - protein, 76 aa, Homo sapiens
AAPAYSRALSRQLSSGVSEIRHTADRWRVSLDVNHFAPDELTVKTKDGVVEITGKHEERQDEHGYISRCFTRKYTL

SEQ ID NO: 9 - protein, 76 aa, Mycobacterium leprae
RFAEQVLGTSARPAVMPMDAWREGEEFVVEFDLPGIKADSLDIDIERNVVTVRARPGVDPDREMRPRGVFNRQLVL

SEQ ID NO: 10 - protein, 76 aa, Mus musculus
PRGPPATARFGVPAEGRSPPPFPGEPWKVCVNVHSFKPEELMVKTKDGYVEVSGKHEEKQQEGGIVSKNFTKKIQL

SEQ ID NO: 11 - protein, 76 aa, Rattus norvegicus
PRGPTATARFGVPAEGRNPPPFPGEPWKVCVNVHSFKPEELMVKTKDGYVEVSGKHEEKQQEGGIVSKNFTKKIQL

SEQ ID NO: 12 - protein, 76 aa, Caenorhabditis elegans
LYPRWAEPIFKEGIDVNSNVVNDDRRFAVDMDCYQFRPEEIQVKTLDDTLMIEGRHEDIRDKDNFTKMYFVRKYQL

SEQ ID NO: 13 - DNA, 19 bp, Homo sapiens
  misc_feature: Fig. 3 control person
ccaccgccaggtttggggt

SEQ ID NO: 14 - DNA, 19 bp, Homo sapiens
  misc_feature: fig. 3 AJ-135
ccaccgccakgtttggggt

SEQ ID NO: 15 - DNA, 21 bp, Homo sapiens
  misc_feature: Fig. 3 second control person
acttcacaaagaaaatccagt

SEQ ID NO: 16 - DNA, 21 bp, Homo sapiens
  misc_feature: fig. 3 CMT-355  and AJ-12
  misc_feature (9)..(9): N can be any nucleotide
acttcacanagaaaatccagt

SEQ ID NO: 17 - DNA, 21 bp, Homo sapiens
  misc_feature: fig. 3 CMT-M and CMT-196
  misc_feature (11)..(11): N can be any nucleotide
acttcacaaanaaaatccagt

SEQ ID NO: 18 - DNA, 20 bp, Artificial Sequence
  Mouse Hsp22 cDNA primer musH11-F
acctttgggtaggtggctct

SEQ ID NO: 19 - DNA, 18 bp, Artificial Sequence
  Mouse Hsp22 cDNA primer musH11-R
gggatgggagcgaagaag

SEQ ID NO: 20 - DNA, 20 bp, Artificial Sequence
  forward primer PRKAB1ex1
ggttgggaaagtgtcggttt

SEQ ID NO: 21 - DNA, 20 bp, Artificial Sequence
  reverse primer PRKAB1ex1
ggagggttcctctcctcaac

SEQ ID NO: 22 - DNA, 20 bp, Artificial Sequence
  forward primer PRKAB1ex2
tccgatcctaaccatgaacc

SEQ ID NO: 23 - DNA, 22 bp, Artificial Sequence
  reverse primer PRKAB1ex2
ttttccactaggcatccatttt

SEQ ID NO: 24 - DNA, 20 bp, Artificial Sequence
  forward primer PRKAB1ex3-4
tctgtagctggtttggcaag

SEQ ID NO: 25 - DNA, 20 bp, Artificial Sequence
  reverse primer PRKAB1ex3-4
agactgtacagcccccacct

SEQ ID NO: 26 - DNA, 20 bp, Artificial Sequence
  forward primer PRKAB1ex5
cttggaaccagtgcatcctt

SEQ ID NO: 27 - DNA, 20 bp, Artificial Sequence
  reverse primer PRKAB1ex5
tttgcaagaggtggacacag

SEQ ID NO: 28 - DNA, 20 bp, Artificial Sequence
  forward primer PRKAB1ex6-7
ggggagaatcttggtttcca

SEQ ID NO: 29 - DNA, 20 bp, Artificial Sequence
  reverse primer PRKAB1ex6-7
accagggcaggtatgaaatg

SEQ ID NO: 30 - DNA, 20 bp, Artificial Sequence
  forward primer SIRT4ex1
tggtgatcaagacagccaag

SEQ ID NO: 31 - DNA, 20 bp, Artificial Sequence
  reverse primer SIRT4ex1
ctgggcaacagagggagact

SEQ ID NO: 32 - DNA, 20 bp, Artificial Sequence
  forward primer SIRT4ex2
cgtctctgacagctttgtgc

SEQ ID NO: 33 - DNA, 20 bp, Artificial Sequence
  reverse primer SIRT4ex2
ctgcacggagaaaagacaca

SEQ ID NO: 34 - DNA, 20 bp, Artificial Sequence
  forward primer SIRT4ex3
ttgggagtcctggagagaca

SEQ ID NO: 35 - DNA, 20 bp, Artificial Sequence
  reverse primer SIRT4ex3
agtatgacccctgtgcaaga

SEQ ID NO: 36 - DNA, 40 bp, Artificial Sequence
  forward primer CITex1
gttggaacccttgggagaacgtgtcacgtgggttcagaaa

SEQ ID NO: 37 - DNA, 20 bp, Artificial Sequence
  reverse primer CITex1
gtgtcacgtgggttcagaaa

SEQ ID NO: 38 - DNA, 40 bp, Artificial Sequence
  forward primer CITex2
tttagcaccaggaggcttgtcccgaccaaagtaatctcca

SEQ ID NO: 39 - DNA, 20 bp, Artificial Sequence
  reverse primer CITex2
cccgaccaaagtaatctcca

SEQ ID NO: 40 - DNA, 20 bp, Artificial Sequence
  forward primer CITex3
aaccatgggacatttttgga

SEQ ID NO: 41 - DNA, 20 bp, Artificial Sequence
  reverse primer CITex3
agagacggaccagccttctt

SEQ ID NO: 42 - DNA, 40 bp, Artificial Sequence
  forward primer CITex4
gacactgtgggaggaggagatctttctccgtgaaggttcg

SEQ ID NO: 43 - DNA, 20 bp, Artificial Sequence
  reverse primer CITex4
tctttctccgtgaaggttcg

SEQ ID NO: 44 - DNA, 40 bp, Artificial Sequence
  forward primer CITex5
cgaagtgctgggattacaggggtgccatgcctgaaattag

SEQ ID NO: 45 - DNA, 20 bp, Artificial Sequence
  reverse primer CITex5
ggtgccatgcctgaaattag

SEQ ID NO: 46 - DNA, 44 bp, Artificial Sequence
  forward primer CITex6
gtccactgagccatgaatgatgactcagtactgtttgtgttgga

SEQ ID NO: 47 - DNA, 24 bp, Artificial Sequence
  reverse primer CITex6
tgactcagtactgtttgtgttgga

SEQ ID NO: 48 - DNA, 40 bp, Artificial Sequence
  forward primer CITex7
acatcaacttggcaatgcacgcttttgtggtttgctcctc

SEQ ID NO: 49 - DNA, 20 bp, Artificial Sequence
  reverse primer CITex7
gcttttgtggtttgctcctc

SEQ ID NO: 50 - DNA, 40 bp, Artificial Sequence
  forward primer CITex8
cttgagctcccaacttcaggctgtgcattgccaagttgat

SEQ ID NO: 51 - DNA, 20 bp, Artificial Sequence
  reverse primer CITex8
ctgtgcattgccaagttgat

SEQ ID NO: 52 - DNA, 40 bp, Artificial Sequence
  forward primer CITex9
cagcttcaacaggggaaaaattccctttctgtgggttgtc

SEQ ID NO: 53 - DNA, 20 bp, Artificial Sequence
  reverse primer CITex9
ttccctttctgtgggttgtc

SEQ ID NO: 54 - DNA, 40 bp, Artificial Sequence
  forward primer CITex10-11
actggggagacctgggttagagaggaagggaagggtccag

SEQ ID NO: 55 - DNA, 20 bp, Artificial Sequence
  reverse primer CITex10-11
agaggaagggaagggtccag

SEQ ID NO: 56 - DNA, 40 bp, Artificial Sequence
  forward primer CITex12-13
agcctgaggggaatcaaaattccccttgttcttgttcctg

SEQ ID NO: 57 - DNA, 20 bp, Artificial Sequence
  reverse primer CITex12-13
tccccttgttcttgttcctg

SEQ ID NO: 58 - DNA, 40 bp, Artificial Sequence
  forward primer CITex14-15
catgaaacgtggcttcaacaggtttctctggatggtttgg

SEQ ID NO: 59 - DNA, 20 bp, Artificial Sequence
  reverse primer CITex14-15
ggtttctctggatggtttgg

SEQ ID NO: 60 - DNA, 40 bp, Artificial Sequence
  forward primer CITex16
acgagctctgtgggaagagatctgtgtggcctcttgtgac

SEQ ID NO: 61 - DNA, 20 bp, Artificial Sequence
  reverse primer CITex16
tctgtgtggcctcttgtgac

SEQ ID NO: 62 - DNA, 40 bp, Artificial Sequence
  forward primer CITex17
cagtgcactttccacactggttcctagttttgccccacag

SEQ ID NO: 63 - DNA, 20 bp, Artificial Sequence
  reverse primer CITex17
ttcctagttttgccccacag

SEQ ID NO: 64 - DNA, 40 bp, Artificial Sequence
  forward primer CITex18-19
tccatgtaccctcctccaacagaacagctgtggaccttgg

SEQ ID NO: 65 - DNA, 20 bp, Artificial Sequence
  reverse primer CITex18-19
agaacagctgtggaccttgg

SEQ ID NO: 66 - DNA, 40 bp, Artificial Sequence
  forward primer CITex20
cggatgcaattcttttccagtgctcctcattcttccatca

SEQ ID NO: 67 - DNA, 20 bp, Artificial Sequence
  reverse primer CITex20
tgctcctcattcttccatca

SEQ ID NO: 68 - DNA, 22 bp, Artificial Sequence
  forward primer CITex21-22
tttgattttccctcttttcacc

SEQ ID NO: 69 - DNA, 22 bp, Artificial Sequence
  reverse primer CITex21-22
tttgattttccctcttttcacc

SEQ ID NO: 70 - DNA, 38 bp, Artificial Sequence
  forward primer CITex23
tcagttccccaagtcactccgtcaaggagggggttgct

SEQ ID NO: 71 - DNA, 18 bp, Artificial Sequence
  reverse primer CITex23
gtcaaggagggggttgct

SEQ ID NO: 72 - DNA, 21 bp, Artificial Sequence
  forward primer CITex24
tgatgatgtggtcgagctaaa

SEQ ID NO: 73 - DNA, 20 bp, Artificial Sequence
  reverse primer CITex24
gtgagcacagcaacttctgg

SEQ ID NO: 74 - DNA, 19 bp, Artificial Sequence
  forward primer HSP22ex1
cagggctgagggctacatc

SEQ ID NO: 75 - DNA, 18 bp, Artificial Sequence
  reverse primer HSP22ex1
gagaggccggctgaactt

SEQ ID NO: 76 - DNA, 20 bp, Artificial Sequence
  forward primer HSP22ex2
agggagagaccccagatcat

SEQ ID NO: 77 - DNA, 20 bp, Artificial Sequence
  reverse primer HSP22ex2
tcatagccagccttggaagt

SEQ ID NO: 78 - DNA, 40 bp, Artificial Sequence
  forward primer HSP22ex3
ccaacattgtatgtccccaaacccgcaccctctaacattt

SEQ ID NO: 79 - DNA, 19 bp, Artificial Sequence
  reverse primer HSP22ex3
cccgcaccctctaacattt